(19) United States
(12) Patent Application Publication
(10) Pub. No.: US 2022/0016438 A1
(43) Pub. Date: Jan. 20, 2022

(54) Systems, methods, and apparatus for a beauty product that can alternatively use RF, ultra red and medical LED light for the purposes of healing acne and growing hair on the scalp

(71) Applicant: Lumia Limited, Hong Kong (CN)

(72) Inventors: Arnold Besraev, Hong Kong (CN); Mickael Sadeh, Hong Kong (CN)

(21) Appl. No.: 17/335,331

(22) Filed: Jun. 1, 2021

Related U.S. Application Data
(60) Provisional application No. 63/052,030, filed on Jul. 15, 2020.

(51) Int. Cl. A61N 5/06; A61N 1/06; A61N 1/32; B08B 1/00; A61H 1/00

(52) U.S. Cl. CPC A61N 5/0617; A61N 1/06; A61N 1/328; B08B 1/006; A61H 1/00; A61N 2005/0651; A61H 2201/0221; A61N 2005/0663; A61N 2005/0644; A61N 2005/0626; A61H 2201/10; A61H 2201/0107; A61H 2201/5082; A61N 2005/0659

(57) ABSTRACT

A system for a beautification product, comprising: a main body that provides vibration and electricity; wherein the main body can attach to a power source, and the main body can have different heads attach to the top of the main body. Also, one of the heads produces RF (radio frequency), one of the heads produces infrared light, and one of the heads produces LED light. The various heads and lights can each heal acne and grow hair on the scalp. Lastly, the heads are interchangeable, and the main body is rechargeable.

### FIELD OF INVENTION

The field of invention for the present disclosure is light therapy to heal skin and grow hair on the scalp.

### BACKGROUND

The background description includes information that may be useful in understanding the present invention. It is not an admission that any of the information provided herein is prior art or relevant to the presently claimed invention, or that any publication specifically or implicitly referenced is prior art.

All publications identified herein are incorporated by reference to the same extent as if each individual publication or patent application were specifically and individually indicated to be incorporated by reference. Where a definition or use of a term in an incorporated reference is inconsistent or contrary to the definition of that term provided herein, the definition of that term provided herein applies and the definition of that term in the reference does not apply. The following description includes information that may be useful in understanding the present invention. It is not an admission that any of the information provided herein is prior art or relevant to the presently claimed invention, or that any publication specifically or implicitly referenced is prior art.

In some embodiments, the numbers expressing quantities of ingredients, properties Such as concentration, reaction conditions, and so forth, used to describe and claim certain embodiments of the invention are to be understood as being modified in some instances by the term “about.”

Accordingly, in some embodiments, the numerical parameters set forth in the written description and attached claims are approximations that can vary depending upon the desired properties sought to be obtained by a particular embodiment.

In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable.

The numerical values presented in some embodiments of the invention may contain certain errors necessarily resulting from the standard deviation found in their respective testing measurements.

Unless the context dictates the contrary, all ranges set forth herein should be interpreted as being inclusive of their endpoints and open-ended ranges should be interpreted to include only commercially practical values. Similarly, all lists of values should be considered as inclusive of intermediate values unless the context indicates the contrary.

As used in the description herein and throughout the claims that follow, the meaning of “a,” “an and “the includes plural reference unless the context clearly dictates otherwise. Also, as used in the description herein, the meaning of “in” includes “in” and “on” unless the context clearly dictates otherwise.

The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g. “Such as”) provided with respect to certain embodiments herein is intended merely to better illuminate the invention and does not pose a limitation on the scope of the invention otherwise claimed.

No language in the specification should be construed as indicating any non-claimed element essential to the practice of the invention.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any Such inclusion or deletion occurs, the specification is herein deemed to contain the group as modified thus fulfilling the written description of all Markush groups used in the appended claims.

In the marketplace today, there are light therapy products that work on the skin. These products have 1 head, that is, they can be used for 1 purpose through 1 method only. This forces every user to purchase a different product for each different purpose. This results in the light therapy process being very expensive.

### SUMMARY

The present disclosure consists of a product that improves the appearance of the skin, and can alternatively grow hair on the scalp. This flexibility is achieved because the product has at least 3 heads that can rotate. This allows the product to provide multiple therapeutic options to the patient.

The patient can be treated through RF light, or through ultra red (infrared) light, or through medical LED light (a regular LED light). These lights serve the purpose of improving the skin, and can have the effect of removing acne. Each light is on a different head, and so when the practitioner or a patient wants to use a different light, the practitioner switches to that head for that light.

This allows the practitioner to utilize 1 device, allowing for ease of carrying and travel, as well as a lower cost than purchasing three separate devices.

There is no other company producing a product that has a chargeable/portable device that can switch heads. This capability will greatly benefit the user by lowering cost because less devices will need to be purchased, as well as conserving space making this product easier to travel with if necessary. The capability will also benefit the user because they can quickly switch heads, instead of perhaps getting an entire new device. Furthermore, each head can be separately replaced in case of damage, as opposed to replacing an entire device.

### BRIEF DESCRIPTION OF DRAWINGS

The patent or application file contains at least one drawing executed in color. Copies of this patent or patent application publication with color drawing(s) will be provided by the Office upon request and payment of the necessary fee.

Many aspects of the present disclosure can be better understood with reference to the attached drawings. The components in the drawings are not necessarily drawn to scale, with emphasis instead being placed upon clearly illustrating the principles of the disclosure. Moreover, in the drawings, like reference numerals designate corresponding parts throughout several views.

FIG. 1A displays one embodiment of the invention, in which the main device is mounted with a head that can produce RF and blue LED light, according to various embodiments of the present disclosure.

FIG. 1B displays one embodiment of the invention, in which the main device is mounted with a head that can produce RF and blue LED light, according to various embodiments of the present disclosure.

FIG. 2 displays another embodiment of the invention, which is a head that can produce RF and blue LED light, according to various embodiments of the present disclosure.

FIG. 3 displays another embodiment of the invention, which is another head that can produce RF, LED and infrared light, according to various embodiments of the present disclosure.

FIG. 4 displays another embodiment of the invention, which is another head. This head is made of silicon, and has a cleansing effect on the skin, according to various embodiments of the present disclosure.

FIG. 5 displays another embodiment of the invention, in which the main device is mounted with a head that can produce red LED light, according to various embodiments of the present disclosure.

FIG. 6 displays another embodiment of the invention, in which the lights on the outer ring and inside ring on the head are turned on.

FIG. 7 displays another embodiment of the invention, in which the lights are off, and daylight reveals that the head of the plate contains holes through which certain lights shine through.

### DETAILED DESCRIPTION

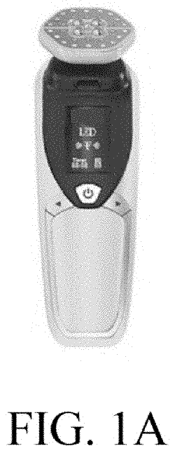

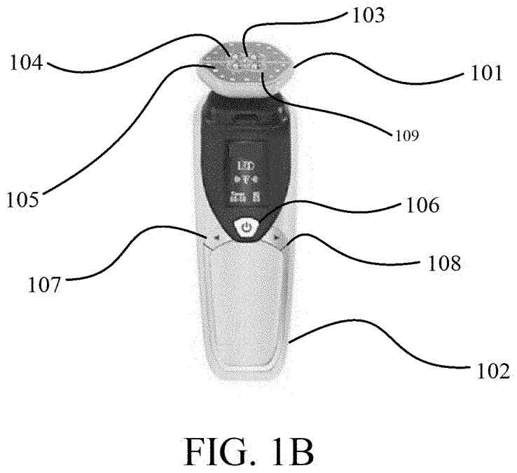

FIG. 1A displays one embodiment of the invention, in which the main device is mounted with a head that can produce RF and blue LED light. The head is designed such that the circle in the middle is a heat sensor. The circle around the heat sensors, i.e. the larger circle, is the control for plus and minus for the rf to close the electric circuit between the skin and the device. The skin is connected to electrodes that respond to the control for plus and minus for the rf.

Furthermore, the 2 plates are also conducting rf. The head is on a smooth and flat surface.

In addition, regarding the 2 plates conducting rf, one plate includes a plus sign and positive current, and the other plate includes a negative sign and negative current. This is because electricity always needs a plus and minus to send the current.

The hole on the plates is for the infrared and medical led heads. The infrared head and the medical led head can alternatively be attached to the main body of the invention.

There is a button to control the power, and buttons to lower and increase the intensity.

The head is shaped in a hexagon, and contains several lights along its edge. The lights along the edge are electrodes.

The lights in the center circle are heat sensors.

FIG. 1B is the same as FIG. 1A, except with identifying lines. The main device 102 has a power button 106, a button 107 to lower intensity, and a button 108 to increase intensity. The head 101 has both RF and blue LED lights. There is a heat sensor 103.

The head 101 is divided into an upper half, which is an upper half plate, and a lower half, which is a lower half plate. These plates are divided by the horizontal line running across head 101. The upper half plate has a positive current, and the lower half plate has a negative current.

The plates are made out of metal.

Radio frequency light 104 is on the head 101, and is produced from 4 protruding half sphere metal objects close to the center of the head 101.

Outside ring lights 105 produce invisible LED lights. These outside ring lights 105 go around the outer edge of head 101.

Inside ring lights 109 produce visual LED lights. These outside ring lights 105 go around the outer edge of head 101.

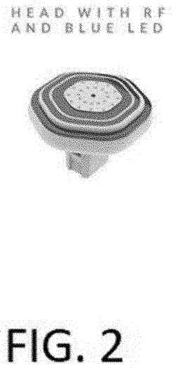

FIG. 2 displays another embodiment of the invention, which is a head that can produce RF and blue LED light.

In the middle there is a heat sensor.

Around the heat sensor there is a blue led light.

Around the blue led light we have 2 circles of rf one plus one minus. This means that one circle has a plus sign and positive current, and the other circle has a negative sign and negative current.

There is a magnetic ring to support a cleaning cloths to take away dirt and bacteria.

The magnetic ring is there to be able to put a cloth to clean the dirt from a user's face. This magnetic ring opens and closes so the user can change the cloth after each use. The magnetic ring supports different types of cleaning cloths, such as a Facial cleaning cloth.

There is an inner circle of multiple blue LED lights, and then there is an outer set of blue LED lights along the edge of the hexagon shaped part of the head. Then there are multiple concentric hexagons alternating in color between dark grey and light grey.

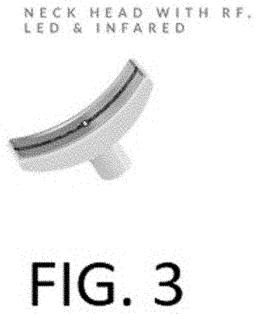

FIG. 3 displays another embodiment of the invention, which is another head that can produce RF, LED and infrared light.

There is a heat sensor in the middle, and the plate of RF has one plus one minus. This means that the plate of RF has a plus sign and positive current, and also has a negative sign and negative current.

Between the plates there are infrared and medical led lights.

The device produces light from the neck and as opposed to the top. The device can alternate between producing RF, LED and infrared light. There is no need to switch to another head in order to produce RF, or LED or infrared light.

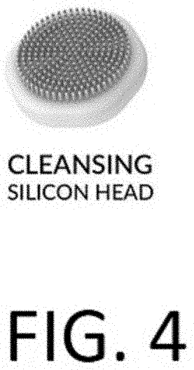

FIG. 4 displays another embodiment of the invention, which is another head. This head is made of silicon, and has a cleansing effect on the skin.

This head goes on top of the normal head of the main body.

On top of the silicon on head there are silicon hair that creates an exfoliation affect when the silicon hair vibrates through the vibration mode on the main body.

The silicon hair consists of silicon strands on the head, that are shaped straight, with a small amount between each hair.

The number of silicon hairs can vary depending on the different skin type that the head is going to be used on. So if the skin is rougher, the number of silicon hairs will be increased, however, if the skin is smoother, then the number of silicon hairs will be decreased.

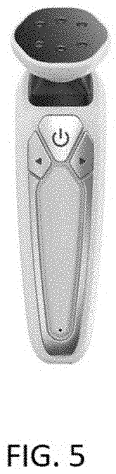

FIG. 5 displays another embodiment of the invention, in which the main device is mounted with a head that can produce red LED light.

The shape allows the device to easily be used around the eyes of a human.

The device heats up, vibrates and has medical led lights for therapeutic effects.

There are 6 infrared lights, 3 in a row, and then 3 below them. There is a button to control the power, and buttons to lower and increase the intensity. The device heats, vibrates and uses infrared lights all together, each function cannot be separated. The highest intensity is the highest that humans can withstand without being harmed.

In another embodiment of the invention, the device is rechargeable, allowing the device to function when not plugged in.

In another embodiment of the invention, the plates are on top of the heads. This is how the rf gets transferred to the skin of the user, because the plate touches the skin.

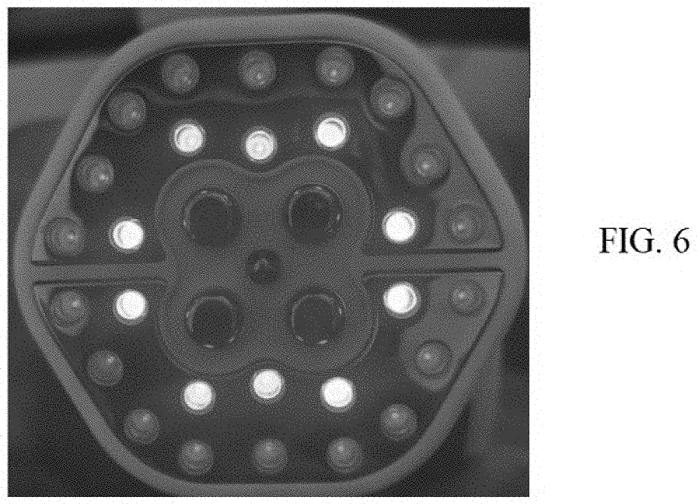

FIG. 6 displays another embodiment of the invention, in which the lights on the outer ring and inside ring on the head are turned on. The lights on the outer ring appear red, but actually produce invisible LED light. The lights on the inside ring appear yellow, and produce visual LED light.

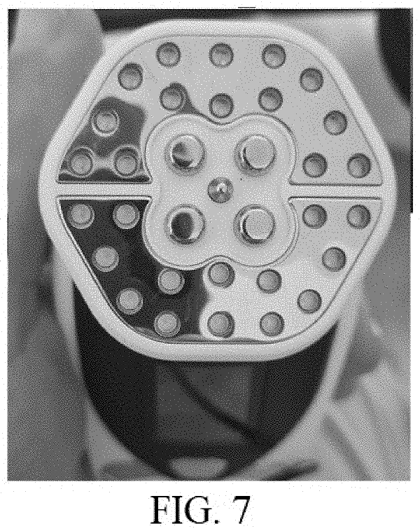

FIG. 7 displays another embodiment of the invention, in which the lights are off, and daylight reveals that the head of the plate contains holes through which certain lights shine through. Specifically, the lights of the outer ring and inside ring shine through the holes.

Having thus described embodiments of the present invention of the present application in detail and by reference to illustrative embodiments thereof, it will be apparent that modifications and variations are possible without departing from the scope of the present invention defined in the appended claims.

1. A system for a beautification product, comprising:
a main body that provides vibration and electricity;
wherein the main body can attach to a power source in order to recharge;
wherein the main body can have different heads attach to the top of the main body;
wherein one of the heads produces RF (radio frequency);
wherein one of the heads produces infrared light;
wherein one of the heads produces LED light;
wherein when the RF head is attached to the main body, the RF can both heal acne and grow hair on the scalp;
wherein when the infrared light head is attached to the main body, the infrared light can both heal acne and grow hair on the scalp;
wherein when the LED light head is attached to the main body, the LED light can both heal acne and grow hair on the scalp;
wherein the heads are interchangeable.

2. The system of claim 1, further comprising:
wherein there is an additional head that can produce both RF and blue LED light;
wherein the top of the additional head is shaped like a hexagon;
wherein the blue LED lights are along all edges of the top of the additional head;
wherein the blue LED lights are also in the shape of a hexagon.

3. The system of claim 1, further comprising:
wherein there is an additional head with 2 plates;
wherein each plate conducts RF;
wherein each plate has a hole for either infrared light or LED light;
wherein the additional head is shaped like a hexagon;
wherein the LED lights are along all edges of the top of the additional head;
wherein the LED lights also in the shape of a hexagon.

4. The system of claim 1, further comprising:
wherein there is an additional head that can produce both RF and blue LED light;
wherein the top of the additional head is shaped like a hexagon;
wherein in the middle of the additional head there is a heat sensor;
wherein there are blue LED lights around the heat sensor;
wherein outside the blue LED lights there are 2 concentric circles that conduct RF;
wherein outside the 2 concentric circles there is a magnetic ring;
wherein the magnetic ring utilizes a cleaning cloth to take away dirt and bacteria from a user's face.

5. The system of claim 1, further comprising:
wherein the main body has an additional head that can produce both RF and blue LED light;
wherein the additional head is shaped like a hexagon;
wherein blue LED lights are along all edges of the top of the additional head;
wherein the top of the additional head is in the shape of a hexagon;
wherein the blue LED lights are also in the shape of a hexagon;
wherein there is a second additional head with 2 plates;
wherein each of the 2 plates on the second additional head conducts RF;
wherein each of the 2 plates on the second additional head has a hole for either infrared light or LED light;
wherein the top of the second additional head is shaped like a hexagon;
wherein the LED lights are along all edges of the top of the second additional head;
wherein the LED lights are also in the shape of a hexagon;
wherein there is a third additional head that can produce both RF and blue LED light;
wherein the top of the third additional head is shaped like a hexagon;
wherein in the middle of the third additional head there is a heat sensor;
wherein the blue LED light is around the heat sensor;
wherein around the blue LED light there are 2 concentric circles that conduct RF;
wherein outside the 2 concentric circles there is a magnetic ring;
wherein the magnetic ring utilizes a cleaning cloth to take away dirt and bacteria from a user's face.

6. A method for beautification, comprising:
providing vibration and electricity through a main body;
wherein the main body is attached to a power source in order to recharge;
wherein the main body has different heads attached to the top of the main body;
wherein one of the heads produces RF (radio frequency);
wherein one of the heads produces infrared light;
wherein one of the heads produces LED light;
wherein when the RF head is attached to the main body, the RF can both heal acne and grow hair on the scalp;
wherein when the infrared light head is attached to the main body, the infrared light can both heal acne and grow hair on the scalp;
wherein when the LED light head is attached to the main body, the LED light can both heal acne and grow hair on the scalp;
wherein the heads are interchangeable.

7. The method of claim 6, further comprising:
wherein the main body has an additional head that can produce both RF and blue LED light;
wherein the additional head is shaped like a hexagon;
wherein blue LED lights are along all edges of the top of the additional head;
wherein the top of the additional head is in the shape of a hexagon;
wherein the blue LED lights are also in the shape of a hexagon.

8. The method of claim 7, further comprising:
wherein there is a second additional head with 2 plates;
wherein each of the 2 plates on the second additional head conducts RF;
wherein each of the 2 plates on the second additional head has a hole for either infrared light or LED light;
wherein the top of the second additional head is shaped like a hexagon;
wherein the LED lights are along all edges of the top of the second additional head;
wherein the LED lights are also in the shape of a hexagon.

9. The method of claim 8, further comprising:
wherein there is a third additional head that can produce both RF and blue LED light;
wherein the top of the third additional head is shaped like a hexagon;
wherein in the middle of the third additional head there is a heat sensor;
wherein the blue LED light is around the heat sensor;
wherein around the blue LED light there are 2 concentric circles that conduct RF;
wherein outside the 2 concentric circles there is a magnetic ring;
wherein the magnetic ring utilizes a cleaning cloth to take away dirt and bacteria from a user's face.

10. A system for a beautification product, comprising:
a main body that provides vibration and electricity;
wherein the main body is attached to a power source in order to recharge;
wherein the main body has different heads attached to the top of the main body;
wherein one of the heads produces RF (radio frequency);
wherein one of the heads produces infrared light;
wherein one of the heads produces LED light;
wherein when the RF head is attached to the main body, the RF can both heal acne and grow hair on the scalp;
wherein when the infrared light head is attached to the main body, the infrared light can both heal acne and grow hair on the scalp;
wherein when the LED light head is attached to the main body, the LED light can both heal acne and grow hair on the scalp;
wherein the heads are interchangeable;
wherein the main body has an additional head that can produce both RF and blue LED light;
wherein the additional head is shaped like a hexagon;
wherein blue LED lights are along all edges of the top of the additional head;
wherein the top of the additional head is in the shape of a hexagon;
wherein the blue LED lights are also in the shape of a hexagon;
wherein there is a second additional head with 2 plates;
wherein each of the 2 plates on the second additional head conducts RF;
wherein each of the 2 plates on the second additional head has a hole for either infrared light or LED light;
wherein the top of the second additional head is shaped like a hexagon;
wherein the LED lights are along all edges of the top of the second additional head;
wherein the LED lights are also in the shape of a hexagon;
wherein there is a third additional head that can produce both RF and blue LED light;
wherein the top of the third additional head is shaped like a hexagon;
wherein in the middle of the third additional head there is a heat sensor;
wherein the blue LED light is around the heat sensor;
wherein around the blue LED light there are 2 concentric circles that conduct RF;
wherein outside the 2 concentric circles there is a magnetic ring;
wherein the magnetic ring utilizes a cleaning cloth to take away dirt and bacteria from a user's face.
